(19) United States
(12) Patent Application Publication
(10) Pub. No.: US 2009/0111733 A1
(43) Pub. Date: Apr. 30, 2009

(54) PAR-4 RELATED METHODS AND COMPOSITIONS

(75) Inventors: Sang Ki Park, Brookline, MA (US); Li-Huei Tsai, Cambridge, MA (US); Yang Shi, Wellesley, MA (US)

Correspondence Address: FOLEY HOAG, LLP;PATENT GROUP (w/HUV HMV), 155 SEAPORT BLVD., BOSTON, MA, 02210-2600, US

(73) Assignee: President and Fellows of Harvard College, Cambridge, MA (US)

(21) Appl. No.: 11/996,028

(22) PCT Filed: Jul. 19, 2006
(86) PCT No.: PCT/US2006/028060

§ 371 Date: Oct. 2, 2008

(51) Int. Cl. A61K 38/00; G01N 33/50; G01N 33/68; A61K 31/7088; A61K 31/22; A61K 31/195; A61K 31/19; A61K 31/192; A61K 31/221; A61P 25/00; C07K 2/00; A01K 67/00

(52) U.S. Cl. 514/2; 435/29; 530/350; 800/9; 514/44; 514/546; 514/551; 514/561; 514/570

(57) ABSTRACT

Provided herein are methods and compositions for treating or preventing mood disorders and certain other mental disorders. Methods may comprise increasing PAR-4 levels or activity and/or the interaction between PAR-4 and the dopamine (D2) receptor

### CROSS-REFERENCE TO RELATED APPLICATIONS

This application claims the benefit of U.S. Provisional Application No. 60/700,266, filed Jul. 18, 2005, the content of which is specifically incorporated by reference herein in its entirety.

### BACKGROUND

Clinical depression is characterized by a combination of symptoms that interfere with the ability to work, study, sleep, eat, and enjoy once pleasurable activities. Symptoms include: persistent sad or anxious mood; feelings of hopelessness or pessimism; feelings of guilt, worthlessness or helplessness; loss of interest in pleasure activities; decreased energy; difficulty concentrating, remembering, or making decisions; sleep abnormalities (e.g. insomnia); appetite and/or weight loss; thoughts of death or suicide; restlessness; and irritability.

Depression is a common disorder, occurring in approximately 10 percent of the U.S. population. Major depression is a leading cause of disability in the U.S. and worldwide, and a leading cause of days lost from work. There are many causes of Clinical Depression having roots in the anatomy of the human brain. Neurotransmitter activity, genetic predisposition, and environmental factors are believed to be involved in the development of depression.

Diagnosis of depression is complicated, requiring a physical examination to rule out certain medications or medical conditions and a psychological examination to thoroughly evaluate the symptoms and determine how severely the symptoms have affected the life of the patient. Depression is difficult to diagnose due to the variety of ways in which depression manifests itself. Moreover, there is no definitive symptom or test to confirm a diagnosis of depression.

The most common treatments involve a combination of psychotherapy and antidepressant medication. There are several types of antidepressant medications available which treat the symptoms of depression, including selective serotonin reuptake inhibitors (SSRIs), tricyclics, and monoamine oxidase inhibitors (MAOIs). SSRIs, a newer class of medications selective for serotonin includes Paxil, Prozac and Zoloft. Tricyclic antidepressants, which include Elavil and Tofranil, work mainly by increasing the level of norepinephline in the brain synapses. Tricyclic antidepressants can cause life threatening heart rhythm disturbances when taken in over-dose, and are contra-indicated in patients with seizure disorders. MAOI, inhibit monoamine oxidase, the main enzyme that breaks down neurochemicals such as norepinephrine, leading to elevated levels of neurotransmitters. MAOIs also impair the breakdown of tyramine, found in some foods, requiring the ingestion of such foods to be prevented in patients taking MAOIs. MAOIs can also interact dangerously with over-the-counter cold and cough medications. These potential dangerous food and drug interactions cause doctors to usually only prescribe MAOIs after other options have failed. Other side effects associated with antidepressant medications include dry mouth, nausea, gastrointestinal problems, weight gain, bladder problems, sexual problems, headache, blurred vision, dizziness, and drowsiness.

Although a variety of medications for depression exist, issues with side effects and compliance make it clear that improved therapies are needed. Current antidepressant medications target the symptoms of depression, and investigation into and treatments aimed at the underlying cause may lead to more pervasive and enduring treatments.

### SUMMARY

Provided herein are methods for identifying agents that modulate the interaction between Par-4 and the dopamine D2 receptor (D2DR). A method may comprise (i) contacting a Par-4 protein, or a portion thereof that is sufficient for interacting with a D2DR protein, with a D2DR protein, or a portion thereof that is sufficient for interacting with a Par-4 protein, in the presence of a test agent; and (ii) determining the level of interaction between the Par-4 protein or portion thereof and the D2DR protein or portion thereof, wherein a different level of interaction between the Par-4 protein or portion thereof and the D2DR protein or portion thereof in the presence of the test agent relative to the absence of the test agent indicates that the test agent is an agent that modulates the interaction between Par-4 and D2DR. A higher level of interaction between the Par-4 protein or portion thereof and the D2DR protein or portion thereof in the presence of the test agent relative to the absence of the test agent indicates that the test agent is an agent that stimulates the interaction between Par-4 and D2DR. A lower level of interaction between the Par-4 protein or portion thereof and the D2DR protein or portion thereof in the presence of the test agent relative to the absence of the test agent indicates that the test agent is an agent that inhibits the interaction between Par-4 and D2DR.

A method for identifying an agent that modulates the interaction between Par-4 and D2DR may also comprise (i) contacting a cell or cell lysate or cell fraction comprising a Par-4 protein, or a portion thereof that is sufficient for interacting with a D2DR protein, and a D2DR protein, or a portion thereof that is sufficient for interacting with a Par-4 protein, with a test agent; and (ii) determining the level of cAMP accumulation or dopamine-dependent cAMP-CREB signaling, wherein a different level of cAMP accumulation or dopamine-dependent cAMP-CREB signaling in the presence of the test agent relative to the absence of the test agent indicates that the test agent is an agent that modulates the interaction between Par-4 and D2DR. A higher or lower level of cAMP accumulation or dopamine-dependent cAMP-CREB signaling in the presence of the test agent relative to the absence of the test agent indicates that the test agent is an agent that inhibits or stimulates, respectively, the interaction between Par-4 and D2DR.

In any of the methods described herein, a cell may comprise a heterologous nucleic acid encoding the Par-4 protein or portion thereof and/or a heterologous nucleic acid encoding the D2DR protein or portion thereof. The cell may be a neuron. The portion of the Par-4 protein may comprise the leucine zipper of Par-4. The Par-4 protein or portion thereof may comprise SEQ ID NO: 2 or a portion thereof. The D2DR protein or portion thereof may comprise the calmodulin binding motif in the third cytoplasmic loop. The D2DR protein or a portion thereof may comprise SEQ ID NO: 4 or a portion thereof. The test agent may be a molecule of a library of molecules. The agent may be a small molecule. A method may further comprise determining the effect of the test agent on the inhibitory tone of D2DR on dopamine-mediated downstream signaling. A method may comprise measuring D2DR-mediated inhibition of forskolin-activated adenylyl cyclase activity in a cell.

In another embodiment, a method for identifying an agent that changes the cellular location of Par-4 in a cell comprises (i) contacting a cell expressing a Par-4 protein or a portion thereof in a first cellular compartment with a test agent; and (ii) determining the cellular location of the Par-4 protein or portion thereof at a certain time after the beginning of the contacting step; wherein a different cellular location of the Par-4 protein or portion thereof in a cell that was contacted with the test agent relative to a cell that was not contacted with the test agent or relative to the cell before contacting it with the test agent, indicates that the test agent is an agent that changes the cellular location of Par-4 in a cell. A method for identifying an agent that enhances nuclear translocation of Par-4 may also comprise (i) contacting a cell expressing a Par-4 protein or a portion thereof in a cellular compartment other than the nucleus; and (ii) determining the cellular location of the Par-4 protein or portion thereof at a certain time after the beginning of the contacting step; wherein the presence of Par-4 or a portion thereof in the nucleus indicates that the test agent is an agent that enhances nuclear translocation of Par-4. The Par-4 protein or a portion thereof may comprise the leucine zipper of the protein. A method for identifying an agent that inhibits nuclear translocation of Par-4 may comprise (i) contacting a cell expressing a Par-4 protein or a portion thereof in the nucleus; and (ii) determining the cellular location of the Par-4 protein or portion thereof at a certain time after the beginning of the contacting step; wherein the presence of Par-4 or a portion thereof in a cellular compartment other than the nucleus indicates that the test agent is an agent that inhibits nuclear translocation of Par-4. The Par-4 protein or a portion thereof may comprise a mutated leucine zipper that is essentially inactive.

Also provided herein are pharmaceutical compositions comprising one or more agents identified by a method described herein. Other compositions comprise an isolated Par-4 protein, or a portion thereof that is sufficient for interacting with a D2DR protein, and an isolated D2DR protein, or a portion thereof that is sufficient for interacting with a Par-4 protein. A composition may further comprise a test agent. Also provided are isolated molecular complexes comprising a Par-4 protein, or a portion thereof that is sufficient for interacting with a D2DR protein, and a D2DR protein, or a portion thereof that is sufficient for interacting with a Par-4 protein.

Further provided herein is an animal model for a Par-4 related disease. A model may comprise or consist of an animal having a mutation in the gene encoding the Par-4 protein, which mutation prevents the encoded Par-4 protein from interacting with the D2DR protein. The Par-4 protein may have a deletion in its leucine zipper region, e.g., rendering it essentially inactive. For example, the Par-4 protein may have a deletion of a portion of or of the entire leucine zipper. An animal may be a mouse.

Other methods described herein include methods for increasing the inhibitory tone on dopamine-mediated downstream signaling in a cell comprising a D2DR protein, comprising, e.g., increasing the level or activity of Par-4 in the cell. The cell may be a neuron. The method may further comprise reducing the level of calcium in the cell.

A method for treating a hypo-active Par-4 related disorder in a subject may comprise increasing the level or activity of Par-4 in cells comprising a D2DR; increasing the interaction between Par-4 and D2DR and/or preventing the nuclear translocation of Par-4 in cells of the subject. A method may also comprise administering to a subject in need thereof, a therapeutically effective amount of a compound of formula I, as further described herein. The disorder may be depression, a depression-like behavior, Parkinson's disease, biopoloar disease, disthymia, eating disorders, restless leg syndrome or hypertension. The method may further comprise administering to the subject an agent that reduces the level of calcium in the cell or prevents the level of calcium in the cell to increase to levels contributing to relieving the inhibitory tone on dopamine-mediated downstream signaling. A method may comprise introducing into the cell a Par-4 protein or portion thereof or a nucleic acid encoding such, such as by administering to the subject a viral vector encoding a Par-4 protein or a portion thereof. A viral vector may be an adenoviral vector or an adenoviral associated vector.

A method for treating a hyper-active Par-4 related disorder in a subject may comprise decreasing the level or activity of Par-4 in cells comprising a D2DR; decreasing the interaction between Par-4 and D2DR and/or stimulating the nuclear translocation of Par-4 in cells of the subject. The disorder may be schizophrenia, schizoaffective disorder, attention deficit hyperactivity disorder (ADHD), Tourette syndrome or drug addition. The method may further comprise administering to the subject an agent that increases the level of calcium in the cell or prevents the level of calcium in the cell to decrease to levels contributing to increasing the inhibitory tone on dopamine-mediated downstream signaling.

Other methods described herein include methods for determining whether a subject has or is likely to develop a hypo-active Par-4 disorder, e.g., comprising determining the cellular location of Par-4 in a neuron of the subject, wherein the presence of Par-4 in the nucleus of the neuron indicates that the subject has or is likely to develop a hypo-active Par-4 disorder.

### BRIEF DESCRIPTION OF THE DRAWINGS

FIG. 1 is a schematic diagram of the human Par-4 protein. Par-4LZ is the protein product encoded by the cDNA clone isolated from the yeast two hybrid screen. Numbers represent corresponding amino acid residues. NLS; nuclear localization signal.

FIG. 2 A shows a series of GST fusion proteins containing D2i3 portions as indicated were generated, purified and used for in vitro binding assays with purified Par-4LZ protein.

FIG. 2B shows the overlap between the Par-4 and calmodulin binding motif in D2i3. Primary sequence of the human D2i3 is shown. Underlined is the region binding to Par-4LZ protein. Bold letters indicate the calmodulin binding motif.

FIG. 3 is a schematic diagram of mouse Par-4 and Par-4ΔLZ proteins.

FIG. 4 shows a model for the involvement of Par-4 in Ca²⁺-dependent regulation of D2DR signaling. A. Complex formation between Par-4 and D2DR is necessary to maintain the inhibitory tone on cAMP signaling generated by D2DR. (B) The Ca²⁺-influx activates calmodulin, shifting the equilibrium toward a calmodulin/D2DR complex. As a result, D2DR efficacy is reduced, thereby relieving D2DR-mediated inhibitory tone on cAMP signaling. (C) Disruption of Par-4/D2DR interaction in Par-4ΔLZ mice facilitates calmodulin/D2DR complex formation upon Ca²⁺ influx, hence an upregulation of dopamine-cAMP signaling including the activation of downstream CREB. CaM; calmodulin. AC; adenylyl cyclase. PKA; cAMP-dependent protein kinase.

### DETAILED DESCRIPTION

Methods for treating depression or depression-like disorders may comprise increasing the protein or activity level of PAR-4 and/or the dopamine D2 receptor or a biologically active analog thereof in a cell of the subject. “PAR-4” refers to “prostate apoptosis response protein”, also referred to as “WT 1-interacting protein”, and “transcriptional repressor PAR4.” The nucleotide and amino acid sequences of the human protein are set forth as SEQ ID NOs: 1 and 2, respectively, and correspond to GenBank Accession Numbers NM_{—}002583 and NP_{—}002574, respectively. A biologically active portion of PAR-4 or a portion that is sufficient for binding to D2DR comprises the leucine zipper domain of the protein, such as about amino acids 245-342 of SEQ ID NO: 2. The dopamine D2 receptor is also referred to as “D2DR”. The sequences of the two human variants of the receptor are set forth in SEQ ID NOs: 3-6 and correspond to GenBank Accession Nos: NM_000795 and NP_000786, respectively for variant 1 and NM_016574 and NP_{—}057658, respectively for variant 2. A biologically active portion of D2DR or a portion that is sufficient for binding to PAR-4 may comprise the third intracellular loop of the long isoform (isoform 1) of the protein, such as about amino acids 212-373 of SEQ ID NO: 6.

The level of protein can be increased in a cell, e.g., by introducing into the cell a nucleic acid encoding the protein operably linked to a transcriptional regulatory sequence directing the expression of the protein in the cell. A protein may have at least about 80%, 90%, 95%, 98% or 99% sequence identity with human PAR-4 or D2DR or a portion thereof. It may also be encoded by a nucleic acid that has at least about 80%, 90%, 95%, 98% or 99% sequence identity with a nucleic acid encoding human PAR-4 or D2DR or a portion thereof. It may also be encoded by a nucleic acid that hybridizes, e.g., under stringent hybridization conditions, to a nucleic acid encoding human PAR-4 or D2DR or a portion thereof.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

A protein may also be a variant of a naturally occurring or wild-type PAR-4 or D2DR protein. A “variant” of a polypeptide refers to a polypeptide having the amino acid sequence of the polypeptide in which is altered in one or more amino acid residues. The variant may have “conservative” changes, wherein a substituted amino acid has similar structural or chemical properties (e.g., replacement of leucine with isoleucine). A variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “variant,” when used in the context of a polynucleotide sequence, may encompass a polynucleotide sequence related to that of a particular gene or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variation is a variation in the polynucleotide sequence of a particular gene between individuals of a given species. Polymorphic variants also may encompass “single nucleotide polymorphisms” (SNPs) in which the polynucleotide sequence varies by one base.

Methods for expressing nucleic acids in cells and appropriate transcriptional regulatory elements for doing so are well known in the art. Alternatively, a protein can be introduced into a cell, usually in the presence of a vector facilitating the entry of the protein into the cells, e.g., liposomes. Proteins can also be linked to transcytosis peptides for that purpose. Yet in other methods, an agent that stimulates expression of the endogenous gene is contacted with a cell. Such agents can be identified as further described herein.

Any means for the introduction of polynucleotides into mammals, human or non- human, or cells thereof may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus, lentivirus, alphavirus, poxvirus, retroviral vectors, vaccinia, HIV, the minute virus of mice, hepatitis B virus, influenza virus or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans. As described in greater detail below, such embodiments of the subject expression constructs are specifically contemplated for use in various in vivo and ex vivo gene therapy protocols.

The expression of a protein, e.g., a PAR-4 or D2DR or a biologically active variant thereof, in cells of a subject to whom, e.g., a nucleic acid encoding the protein was administered, can be determined, e.g., by obtaining a sample of the cells of the patient and determining the level of the protein in the sample, relative to a control sample.

In another embodiment, a protein or biologically active variant thereof, is administered to the subject such that it reaches the target cells, and traverses the cellular membrane. Polypeptides can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Robert K Scopes “Protein Purification: Principles and Practice” Third Ed. Springer-Verlag, N.Y. 1994). In one embodiment, the polypeptide is produced as a fusion polypeptide comprising an epitope tag consisting of about six consecutive histidine residues. The fusion polypeptide can then be purified on a Ni⁺⁺ column. By inserting a protease site between the tag and the polypeptide, the tag can be removed after purification of the peptide on the Ni⁺⁺ column. These methods are well known in the art and commercial vectors and affinity matrices are commercially available.

Administration of polypeptides can be done by mixing them with liposomes, as described above. The surface of the liposomes can be modified by adding molecules that will target the liposome to the desired physiological location.

In one embodiment, a protein is modified so that its rate of traversing the cellular membrane is increased. For example, the polypeptide can be fused to a second peptide which promotes “transcytosis,” e.g., uptake of the peptide by cells. In one embodiment, the peptide is a portion of the HIV transactivator (TAT) protein, such as the fragment corresponding to residues 37-62 or 48-60 of TAT, portions which are rapidly taken up by cell in vitro (Green and Loewenstein, (1989) Cell 55:1179-1188). In another embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example Derossi et al. (1996) J Biol Chem 271:18188-18193; Derossi et al. (1994) J Biol Chem 269:10444-10450; and Perez et al. (1992) J Cell Sci 102:717-722.

The term “treating” is art-recognized and refers to curing as well as ameliorating at least one symptom of any condition or disease or preventing a condition or disease from worsening. A treatment may be prophylactic or therapeutic. A “patient,” “subject” or “host” to be treated by the subject method may mean either a human or non-human animal, e.g., a mammal. Exemplary mammals include humans, primates, bovines, porcines, canines, felines, and rodents (e.g., mice and rats).

Small molecules or agents that modulate, e.g., enhance, Par-4 expression can also be used. For example, ionomycin or glutamate may be used. In addition, Valproate or valproic acid, derivatives and analogs thereof may be used for treating any of the diseases that would benefit from increasing Par-4 expression or the interaction between Par-4 and D2DR. Exemplary derivatives and analogs of valproate are compounds of formula I, wherein formula I is represented by:

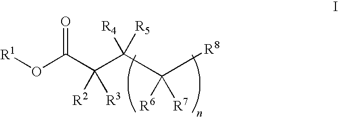

or a pharmaceutically acceptable salt thereof;

wherein,

R¹ is H, alkyl, heteroalkyl, allyl, aryl, or aralkyl;

R², R⁴, and R⁶ each represent independently for each occurrence H, alkyl, heteroalkyl, allyl, aryl, aralkyl, halogen, hydroxyl, alkoxy, —N(R⁹)₂, —C(O)R⁹, —OC(O)R⁹, —CO₂R⁹, —C(O)N(R⁹)₂, or —N(R⁹)C(O)R⁹;

R³, R⁵, R⁷, and R⁸ each represent independently for each occurrence H, alkyl, heteroalkyl, allyl, aryl, aralkyl, or alkoxy;

R⁹ represents independently for each occurrence H, alkyl, aryl, or aralkyl;

n is 1, 2, 3, 4, 5, 6, 7, or 8; and

provided that at least one of R², R³, R⁴ or R⁵ is alkyl.

In certain embodiments, R¹ is H or alkyl; R², R³, R⁴, R⁵, R⁶, R⁷, and R⁸ each represent independently for each occurrence H, alkyl, heteroalkyl, allyl, aryl, or aralkyl. In certain embodiments, R², R³, R⁴, R⁵, R⁶, R⁷, and R⁸ each represent independently for each occurrence H, alkyl, or aralkyl. In certain embodiments, R², R³, R⁴, R⁵, R⁶, R⁷, and R⁸ each represent independently for each occurrence H or alkyl. In certain embodiments, R¹, R², R³, R⁴, R⁵, R⁶, R⁷, and R⁸ each represent independently for each occurrence H or alkyl. In certain embodiments, n is 2; and R¹, R², R³, R⁴, R⁵, R⁶, R⁷, and R⁸ each represent independently for each occurrence H or alkyl. In certain embodiments, R² is (C₁-C₆)alkyl; and R¹, R³, R⁴, R⁵, R⁶, R⁷, and R⁸ each represent independently for each occurrence H or alkyl. In certain embodiments, n is 2; R² is (C₁-C₆)alkyl; and R¹, R³, R⁴, R⁵, R⁶, R⁷, and R⁸ each represent independently for each occurrence H or alkyl. In certain embodiments, said pharmaceutically acceptable salt is a sodium, lithium, potassium, calcium, or magnesium salt. In certain embodiments, said compound is one of the following:

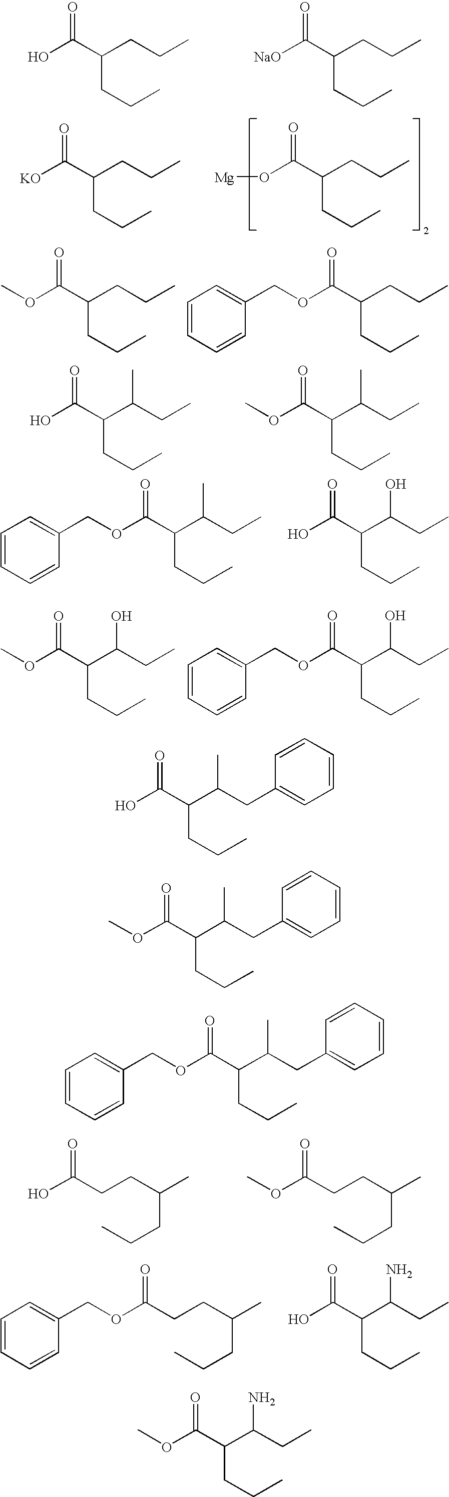

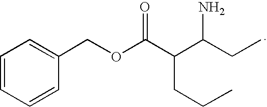

In one embodiment, the compound is

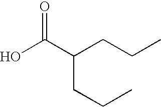

or a pharmaceutically acceptable salt thereof. In another embodiment, the compound is one of the following:

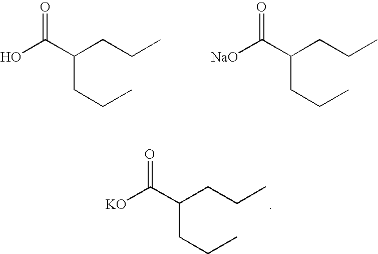

In another embodiment, the compound is

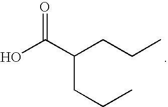

.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain allcyl groups, branched-chain allcyl groups, cycloallcyl (alicyclic) groups, alkyl substituted cycloallcyl groups, and cycloallcyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloallcynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloallcyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

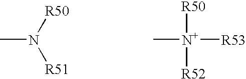

wherein R50, R51 and R52 each independently represent a hydrogen, an alkyl, an alkenyl, —(CH₂)m-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ-R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

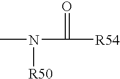

wherein R50 is as defined above, and R54 represents a hydrogen, an alkyl, an alkenyl or —(CH₂)ₘ-R61, where m and R61 are as defined above.

The term “amido” is art recognized as an amino-substituted carbonyl and includes a moiety that may be represented by the general formula:

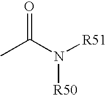

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of -S-alkyl, -S-alkenyl, -S-alkynyl, and -S-(CH₂)ₘ-R61, wherein m and R61 are defined above. Representative alkylthio groups include methylthio, ethyl thio, and the like.

The term “carboxyl” is art recognized and includes such moieties as may be represented by the general formulas:

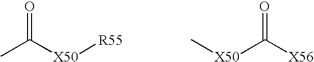

wherein X50 is a bond or represents an oxygen or a sulfur, and R55 and R56 represents a hydrogen, an alkyl, an alkenyl, —(CH₂)ₘ-R6 1 or a pharmaceutically acceptable salt, R56 represents a hydrogen, an alkyl, an alkenyl or —(CH2)ₘ-R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O-(CH₂)ₘ-R61, where m and R61 are described above.

The term “sulfonate” is art recognized and refers to a moiety that may be represented by the general formula:

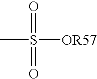

in which R57 is an electron pair, hydrogen, alkyl, cycloalkyl, or aryl.

The term “sulfate” is art recognized and includes a moiety that may be represented by the general formula:

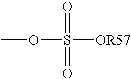

in which R57 is as defined above.

The term “sulfonamido” is art recognized and includes a moiety that may be represented by the general formula:

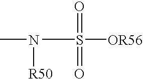

in which R50 and R56 are as defined above.

The term “sulfamoyl” is art-recognized and refers to a moiety that may be represented by the general formula:

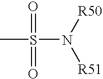

in which R50 and R51 are as defined above.

The term “sulfonyl” is art-recognized and refers to a moiety that may be represented by the general formula:

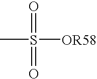

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

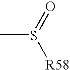

in which R58 is defined above.

The term “phosphoryl” is art-recognized and may in general be represented by the formula:

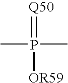

wherein Q50 represents S or O, and R59 represents hydrogen, a lower alkyl or an aryl. When used to substitute, e.g., an alkyl, the phosphoryl group of the phosphorylalkyl may be represented by the general formulas:

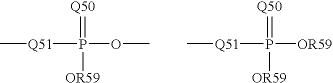

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

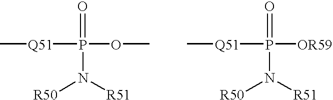

wherein Q51, R50, R51 and R59 are as defined above.

The term “phosphonamidite” is art-recognized and may be represented in the general formulas:

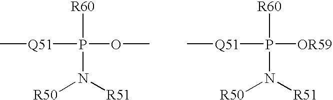

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, tliioalkenyls, thioallcynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se-(CH₂)ₘ-R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an allcyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of this invention, the chemical elements are identified in accordance with the Periodic Table of the Elements, CAS version, Handbook of Chemistry and Physics, 67th Ed., 1986-87, inside cover.

Any disease relating to an abnormal PAR-4 or D2DR activity or level may be treated as described herein. A “hypo-active PAR-4 related disorder” includes diseases, disorders and conditions that are associated with a lower than normal dopamine-mediated downstream signaling. Exemplary diseases include depression, a depression-like behavior, Parkinson's disease, biopoloar disease, disthymia, eating disorders, restless leg syndrome and hypertension.

A “hyper-active PAR-4 related disorder” includes diseases, disorders and conditions that are associated with a higher than normal dopamine-mediated downstream signaling. Exemplary diseases include schizophrenia, schizoaffective disorder, attention deficit hyperactivity disorder (ADHD), Tourette syndrome and drug addition.

Other diseases that may be treated with agents that increase PAR-4 activity or level include cancer (see, e.g., Ranganathan et al. (2005) Ann NY Acad Sci. 2005 Nov;1059:76). Exemplary cancers include carcinomas, e.g., basal cell carcinomas, squamous cell carcinomas, carcinosarcomas, adenocystic carcinomas, epidermoid carcinomas, nasopharyngeal carcinomas, renal cell carcinomas, papillomas, and epidermoidomas. Exemplary cancers are those of the brain including glioblastomas, medulloblastoma, astrocytoma, oligodendroglioma, ependymomas; kidney; colon; lung; liver; pancreas; endometrium; spleen; small intestine; stomach; skin; head and neck; esophagus; hormone-dependent cancers including breast, prostate, testicular, and ovarian cancers; lymphomas (lymph node); and leukemias including cancer of blood cells and bone marrow. Other examples of cancers that can be treated include acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondrosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, and Wilm's tumor.

Generally, because PAR-4 is a pro-apoptotic protein, an increase in its protein level or activity may be beneficial in the treatment of diseases in which one wishes to kill certain cells, e.g., proliferative cell diseases. In addition to malignant cancer, other types of proliferative disorders that can be treated according to the invention include non malignant cell proliferative disorders, e.g., benign cancers, neurofibromatosis; glaucoma; psoriasis; rheumatoid arthritis; restenosis; inflammatory bowel disease; chemotherapy-induced alopecia and mucositis; keratoacanthoma and actinic keratosis; smooth muscle cell hyper-proliferation, e.g., in atherosclerosis and restenosis; inhibiting vascularization, e.g., in tumors; cell hyper-proliferations stimulated by, e.g., hepatitis C or delta and related viruses, and papilloma viruses (HPV); hyperplastic epidermal conditions, such as keratosis; autoimmune diseases; atopic dermatosis; dermatitis; lens epithelial cell proliferation, e.g., to prevent post-operative complications of extracapsular cataract extraction; comeopathies, e.g., marked by corneal epithelial cell proliferation, as for example in ocular epithelial disorders such as epithelial downgrowth or squamous cell carcinomas of the ocular surface; trichosis, e.g. hypertrichosis; hirsutism; inflammatory diseases; infectious diseases; asthma, allergies, e.g., allergic rhinitis; excema; fibromas; and warts The methods described herein may be used for treating or preventing proliferative skin disorders, e.g., any disease/disorder of the skin marked by unwanted or aberrant proliferation of cutaneous tissue, e.g., X-linked ichthyosis, psoriasis, atopic dermatitis, allergic contact dermatitis, epidermolytic hyperkeratosis, epidermodysplasia, epidermolysis, and seborrheic dermatitis.

Examples of autoimmune diseases that may be treated or prevented as described herein include active chronic hepatitis, addison's disease, anti-phospholipid syndrome, atopic allergy, autoimmune atrophic gastritis, achlorhydra autoimmune, celiac disease, crohn's disease, cushing's syndrome, dermatomyositis, diabetes (type I), discoid lupus, erythematosis, goodpasture's syndrome, grave's disease, hashimoto's thyroiditis, idiopathic adrenal atrophy, idiopathic thrombocytopenia, insulin-dependent diabetes, lambert-eaton syndrome, lupoid hepatitis, some cases of lymphopenia, mixed connective tissue disease, multiple sclerosis, pemphigoid, pemphigus vulgaris, pernicious anema, phacogenic uveitis, polyarteritis nodosa, polyglandular auto. syndromes, primary biliary cirrhosis, primary sclerosing cholangitis, psoriasis, raynaud's syndrome, reiter's syndrome, relapsing polychondritis, rheumatoid arthritis, schmidt's syndrome, limited scleroderma (or crest syndrome), severe combined immunodeficiency syndrome (SCID), sjogren's syndrome, sympathetic ophthalmia, systemic lupus erythematosis, takayasu's arteritis, temporal arteritis, thyrotoxicosis, type b insulin resistance, ulcerative colitis and wegener's granulomatosis, in which it is desirable to eliminate autoimmune cells.

Compounds, nucleic acids, proteins, cells and other compositions can be administered to a subject according to methods known in the art. For example, nucleic acids encoding a protein or an antisense molecule can be administered to a subject as described above, e.g., using a viral vector. Cells can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Pharmaceutical agents for use in accordance with the present methods may be formulated in conventional manner using one or more physiologically acceptable carriers or excipients. Thus, proteins and nucleic acids described herein as well as compounds or agents that increase the protein or expression level of nucleic acids described herein, and their physiologically acceptable salts and solvates may be formulated for administration by, for example, injection, inhalation or insufflation (either through the mouth or the nose) or oral, buccal, parenteral or rectal administration. In one embodiment, the agent is administered locally, e.g., at the site where the target cells are present, such as by the use of a patch.

Agents can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

Animal-based disease systems, such as those described herein, may be used to identify compounds capable of ameliorating disease symptoms. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the disease or phenotype. Neonatal, juvenile, and adult animals can also be exposed.

More particularly, using an animal model described herein, methods of identifying agents are provided, in which such agents can be identified on the basis of their ability to affect at least one phenotype associated with a PAR-4 function or dysfunction. In one embodiment, the present invention provides a method of identifying agents that modulate PAR-4 function or level of its interaction with D2DR. The method may include measuring a physiological response of the animal, for example, to the agent, and comparing the physiological response of such animal to a control animal, wherein the physiological response of the animal described herein as compared to the control animal indicates the specificity of the agent. A “physiological response” is any biological or physical parameter of an animal that can be measured.

Also provided herein are screening assays for identifying agents that modulate the interaction between Par-4 and D2DR or agents that increase the protein level or activity of Par-4. Methods for identifying agents that modulate the interaction between Par-4 and the dopamine D2 receptor (D2DR). Screening methods may be cell free or cell based. In one embodiment, a method comprises (i) contacting a Par-4 protein, or a portion thereof that is sufficient for interacting with a D2DR protein, with a D2DR protein, or a portion thereof that is sufficient for interacting with a Par-4 protein, in the presence of a test agent; and (ii) determining the level of interaction between the Par-4 protein or portion thereof and the D2DR protein or portion thereof, wherein a different level of interaction between the Par-4 protein or portion thereof and the D2DR protein or portion thereof in the presence of the test agent relative to the absence of the test agent indicates that the test agent is an agent that modulates the interaction between Par-4 and D2DR. An agent stimulates or inhibits the interaction between Par-4 and D2DR if a higher or lower, respectively, level of interaction between the Par-4 protein or portion thereof and the D2DR protein or portion thereof is observed in the presence of the test agent relative to the absence of the test agent.

A method for identifying an agent that modulates the interaction between Par-4 and D2DR may also comprise (i) contacting a cell comprising a Par-4 protein, or a portion thereof that is sufficient for interacting with a D2DR protein, and a D2DR protein, or a portion thereof that is sufficient for interacting with a Par-4 protein, with a test agent; and (ii) determining the level of cAMP accumulation or dopamine-dependent cAMP-CREB signaling, wherein a different level of cAMP accumulation or dopamine-dependent cAMP-CREB signaling in the presence of the test agent relative to the absence of the test agent indicates that the test agent is an agent that modulates the interaction between Par-4 and D2DR. A lower level of cAMP accumulation or dopamine-dependent cAMP-CREB signaling in the presence of the test agent relative to the absence of the test agent indicates that the test agent is an agent that stimulates the interaction between Par-4 and D2DR.

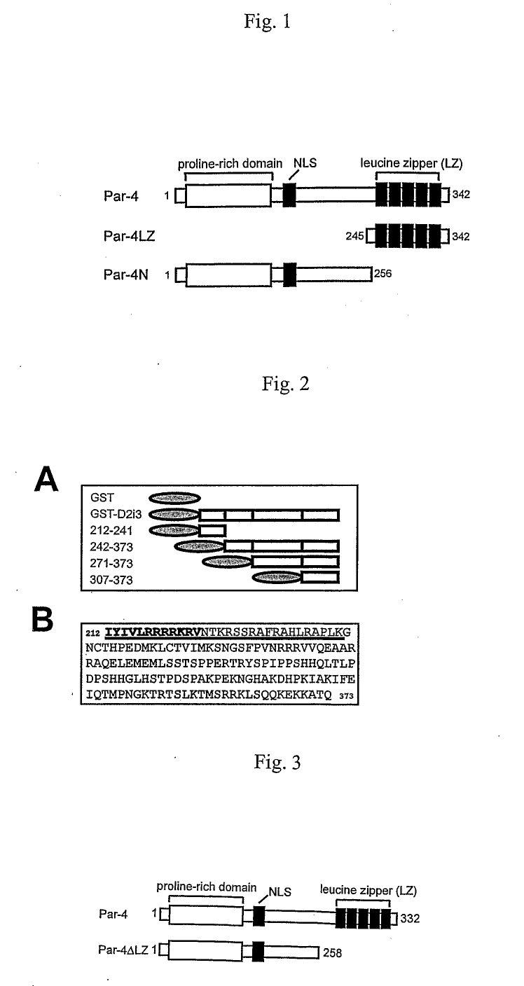

Other screening assays for identifying a novel class of antidepressants and/or mood stabilizers are based on the nuclear translocation of Par-4 as readout. As readout of the effect of small molecules on Par-4 we will use a characteristic feature of Par-4, shuttling between cytoplasmic and nuclear compartments, which can be easily monitored by fluorescence microscopy. The relevance of usage of the Par-4 nuclear shuttling in the screening is supported by the observation that activation of glutamate receptors using glutamate can induce nuclear translocation in the cultured striatal neurons. Given that glutamate is a physiological Ca²⁺ mobilizer in the neuron, a mechanistic linkage between Ca²⁺-mediated downregulation of Par-4/D2DR interaction (see Example 1) and the nuclear translocation of Par-4 is highly likely. Moreover, we also observed that a deletion of Par-4 in the C-terminus (Par-4ΔLZ), an equivalent of truncated Par-4 expressed in the Par-4ΔLZ, is more preferentially localized to nuclei in N2a and HEK293 cells (FIG. 2), further supporting the idea that the depressive phenotypes of Par-4ΔLZ mice is associated with preferential nuclear location of Par-4. Thus, a molecule that has an activity to alter nuclear translocation of Par-4 in the cell may have potential as antidepressants and/or mood-stabilizing drugs.

One assay is a cell-based assay using Par-4 nuclear translocation as readout of the activity of the small molecules. To monitor intracellular location of Par-4, the enhanced green fluorescence protein (EGFP) may be fused either to full-length Par-4 (EGFP-Par-4) or to Par-4ΔLZ proteins (EGFP-Par-4ΔLZ). Stable cell lines expressing either EGFP-Par-4 or EGFP-Par-4ΔLZ may be constructed, e.g., in the human embryonic kidney 293 cells (HEK293). A EGFP-Par-4/HEK293 cell line may be suitable for screening of small molecules that enhance the nuclear location of Par-4. Conversely, the EGFP-Par-4ΔLZ/HEK293 can be used in the screening for the small molecules that block the nuclear translocation of Par-4. In the screening, the cells may be plated in the multi-well culture dish, cultured for 1-2 days and treated with small molecule libraries. Any molecules that elicits altered localization of Par-4 or Par-4ΔLZ proteins are agents that modulate Par-4 activity and can be used for treating or preventing associated diseases.

Any identified agents, such as small molecules may be tested in mouse depression-like paradigms such as Porsolt's forced swim test, tail suspension test and novelty suppressed feeding test. Molecules that exhibit changes in behavioral activity of the mice tested have high potential as antidepressants and/or mood stabilizing drugs.

Most of the current antidepressants (tricyclics or SSRIs) are blockers of monoamine transporters that reside on the plasma membrane of presynaptic monoaminergic neurons. The efficacy of those antidepressants are primarily attributed to the acute increase in monoamine neurotransmitters, mainly 5-HT in the synaptic cleft, and secondarily to undefined adaptation of affected systems. In this regard, the uniqueness of the putative antidepressants targeting Par-4 is two folds. First, given that Par-4 is a novel modulator of D2DR signaling, the antidepressants targeting Par-4 will display their efficacy through modulating dopamine system. Second, as Par-4 is expressed intracellularly the putative antidepressants will show an efficacy by directly modulating intracellular signaling involving Par-4 function. The agents, such as small molecules, obtained in screening assays, e.g., as described herein, may have commercial potential as antidepressants, mood stabilizers and/or reagent as a Par-4 related research reagent.

Modulation, e.g., inhibition or stimulation, may be by a factor of about 50%, 2 fold, 3 fold, 5 fold, 10 fold, 25 fold, 50 fold, 100 fold or more.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The present invention is further illustrated by the following examples which should not be construed as limiting in any way. The contents of all cited references (including literature references, issued patents, published patent applications and GenBank Accession numbers as cited throughout this application) are hereby expressly incorporated by reference.

### EXAMPLES

### Example 1

### Par-4 Links Dopamine Signaling and Depression

The figures corresponding to this example are set forth in Park et al. (2005) Cell 122:275.

### SUMMARY

Prostate apoptosis response 4 (Par-4) is a leucine zipper containing protein that plays a role in apoptosis. Although Par-4 is expressed in neurons, its physiological role in the nervous system is unknown. Here we identify Par-4 as a regulatory component in dopamine signaling. Par-4 directly interacts with the dopamine D2 receptor (D2DR) via the calmodulin binding motif in the third cytoplasmic loop. Calmodulin can effectively compete with Par-4 binding in a Ca²⁺-dependent manner, providing a novel route for Ca²⁺-mediated down-regulation of D2DR efficacy. To examine the importance of the Par-4/D2DR interaction in dopamine signaling in vivo, we used a mutant mouse lacking the D2DR interaction domain of Par-4, Par-4ΔLZ. Primary neurons from Par-4ΔLZ embryos exhibit enhanced dopamine-cAMP-CREB signaling pathway, indicating an impairment in dopamine signaling in these cells. Remarkably, Par-4ΔLZ mice display significantly increased depression-like behaviors. Collectively, these results provide evidence that Par-4 constitutes a unique molecular link between impaired dopamine signaling and depression.

### Introduction

Depression, characterized mainly by low mood, a motivation, anhedonia, low energy and/or fatigue, is one of the most prevalent disorders with the estimated lifetime prevalence of 16.2% in the US adult population (Blazer et al., 1994), resulting in tremendous social costs (Greenberg et al., 1993). Although the cause of depression is obviously multifaceted, the “monoamine hypothesis” describing deficiency or imbalance of the monoamine systems as the cause has been a central topic of research (Bunney and Davis, 1965; Coppen, 1967; Schildlraut et al., 1965). The hypothesis was initiated and supported by the fact that most of the antidepressants share the property of acutely modifying the serotonin or noradrenaline levels at the synapse (Delay et al., 1952; Fuller, 1995; Kuhn, 1958; Leonard, 1978). However, since clinical effects of antidepressants are usually significantly delayed, it is now believed that an adaptation of downstream events, including changes in gene expression and/or modification of other neurotransmitter systems, by chronic treatment underlies their antidepressant efficacy (Manji et al., 2001; Wong and Licinio, 2001). Moreover, a large fraction of depressive subjects is resistant to the current antidepressant therapies (Baldessarini, 1989), demanding improvement of the therapeutic strategies.

Modulating the brain's reward and motivation circuits, mainly governed by dopamine, has been one of the attractive targets for treating depressive disorders (Kinney, 1985). Dopamine exerts its function in target cells through five known subtypes of dopamine receptors (D1, 2, 3, 4 and 5) to regulate motor control, stereotypic behaviors, arousal, mood, motivation, and endocrine function (Missale et al., 1998). Dopamine D2 receptor (D2DR), the predominant D2-like dopamine receptor subtype, is coupled to the inhibitory G-protein (Gi) to downregulate cAMP signaling upon activation (De Camilli et al., 1979). Impairment in the function of D2DR is implicated in various psychiatric disorders such as schizophrenia, mood disorders, and drug addiction (Nestler, 2001). Understanding the details of the modulatory events in D2DR-mediated intracellular signaling is believed to provide novel therapeutic targets for treating various associated disorders.

Prostate apoptosis response 4 (Par-4) is a leucine zipper containing protein that was initially identified as a proapoptotic factor induced by apoptotic stimuli (Sells et al., 1994). Par-4 interacts with PKCξ (Diaz-Meco et al., 1996) to interfere with the prosurvival activity of NFκB (Diaz-Meco et al., 1999). Par-4 also interacts with Wilms' tumor 1 (WT1) to inhibit the growth arrest induced by WT1 (Johnstone et al., 1996). In the nervous system, Par-4 induction has been linked to neuronal death in variouse neurodegenerative diseases (Duan et al., 1999b; Guo et al., 1998; Pedersen et al., 2000). Although Par-4 is prominently detected in synaptic compartments of the brain (Duan et al., 1999a), a physiological role for Par-4 in differentiated neurons has not been elucidated. In the present study, we identify Par-4 as a novel modulator for Ca²⁺-dependent regulation of D2DR signaling. Based on behavioral abnormalities observed in mice with disrupted Par-4/D2DR interaction, we propose that Par-4 constitutes a missing link between D2DR signaling and the manifestation of depressive symptoms.

### RESULTS

PAR-4 Directly Interacts with D2DR

To better understand the mechanistic details behind D2DR-mediated signaling we attempted to discover novel modulatory components for D2DR-mediated intracellular signaling by exploring D2DR-interacting proteins. We identified prostate apoptosis response 4, Par-4, as a D2DR-interacting protein in a yeast two hybrid screen using a human fetal embryonic brain library and the third intracellular loop of the long isoform of human D2DR (D2i3, amino acid residues 212-373) as bait. The human Par-4 cDNA clone recovered from the yeast two hybrid screen encompasses amino acid residues 245-342 that harbor the leucine zipper domain of Par-4 (Par-4LZ, FIG. 1). The LZ domain-dependent interaction was further verified by the prominent interaction-dependent growth on His⁻/Ura⁻ media and by P-galactosidase expression. The direct interaction of Par-4 with D2i3 was demonstrated by an in vitro binding assay using purified GST-D2i3 and Par-4LZ proteins. Approximately 50% of the total Par-4LZ protein was pulled down by an equimolar amount of GST-D2i3 protein. Importantly, the endogenous D2DR and Par-4 can be communoprecipitated from mouse brain lysate, suggesting that the two proteins potentially form a functional complex in vivo. Interestingly, the communoprecitation revealed a predominant signal of ˜48 kD that corresponds to the proposedly monomeric, un- or mildly glycosylated D2DR species (Fishburn et al., 1995; Jarvie and Niznik, 1989). Although there is the possibility of selective enrichment in the sample preparation procedure and/or preferential detection of the D2DR species, given that D2DR exists in differentially modified states in vivo, this result may suggest a functionally selective interaction of Par-4 with a subspecies of D2DR in vivo, which is yet to be investigated.

To assess relative specificity of D2DR and Par-4 interaction, we tested if Par-4 interacts with other structurally and functionally related G-protein-coupled receptors, including the dopamine D3 receptor (D3DR), the 5-hydroxytryptamine (serotonin) receptors (5 HTR) 1A, 1B, 2A and 2B and the α-adrenergic receptor 2A (αAR2A. In yeast two hybrid assays, no significant interaction-dependent marker expression was detected in the Par-4LZ and receptor construct cotransformants, indicating that Par-4 interaction with D2DR is relatively specific.

We next examined Par-4 expression in the CNS. The western blot analyses revealed that Par-4 is expressed in various brain regions, including the striatum, cortex, thalamus, hippocampus, cerebellum and nigra. We examined whether Par-4 is expressed in the medium spiny neurons in the striatum, in which most of the dopaminergic inputs are processed (Murer et al., 2002). Indeed, Par-4 is detected in the DARPP-32-positive medium spiny neurons in mouse striatal sections (Ouimet et al., 1998). Next, we examined whether D2DR and Par-4 coexpressed in the same cells in the striatum. 90.3±2.6% and 82.7±1.7% of striatal neurons expressed detectable levels of D2DR and Par-4, respectively. The level of Par-4 expression appears variable in different cell types. The Par-4 positive cells were mostly D2DR-positive (˜97%), demonstrating that Par-4 indeed is expressed in D2DR-positive neurons in the striatum. Furthermore, D2DR, Par-4 and synaptophysin colocalize in cultured striatal neurons. The colocalization is detected primarily in the periphery of the cell soma and neuronal processes, where the main pool of functional D2DR is localized (Hersch et al., 1995). Consistently, Par-4 and D2DR co-fractionated in the synaptosomal fraction. Taken together, these results strongly suggest a physiological role for Par-4 in D2DR-mediated dopamine signaling in the striatum, which is likely conferred by its direct interaction with D2i3.

Calmodulin Competes with PAR-4 in Binding to D2I3 in a CA²⁺-Dependent Manner

The binding domain of D2DR to Par-4 was localized to the first 30 amino acid residues of D2i3 as indicated by in vitro binding assays (FIGS. 2A and 2B). Intriguingly, the binding region (amino acid residues 212-241) harbors the site known to interact with calmodulin (Bofill-Cardona et al., 2000). Indeed, calmodulin binds to D2i3 in a Ca²⁺-dependent manner whereas Par-4LZ binding is constitutive regardless of the presence of Ca²⁺. To determine whether Par-4 and calmodulin compete for binding to D2i3, we examined the association of Par-4LZ protein with D2i3 in the presence of increasing calmodulin levels. The interaction of Par-4LZ with D2i3 was effectively interfered with an increased binding of calmodulin in the presence of Ca²⁺, indicating that calmodulin can displace Par-4 from D2i3 in a Ca²⁺ dependent manner. Consistent with the in vitro binding experiments, the co-immunoprecipitation of Par-4 with D2DR-EGFP was significantly reduced in the presence of either ionomycin, a Ca²⁺ ionophore, or thapsigargin, an intracellular Ca²⁺ mobilizer (Lytton et al., 1991) in the stable D2DR-EGFP cell line, indicating that the Par-4/D2DR association can be downregulated by increased Ca²⁺ in the cellular context.

### PAR-4 Loss of Function Downregulates D2DR Efficacy to Reduce Inhibitory Tone on Dopamine-Mediated Camp Signaling

It has been reported previously that calmodulin binding to D2i3 negatively regulates D2DR by interfering with the coupling of the Gi-protein in a noncompetitive manner (Bofill-Cardona et al., 2000). Thus, shift of an equilibrium from the Par-4/D2DR interaction to the calmodulin/D2DR interaction by augmented Ca²⁺ concentrations most likely results in a downregulation of D2DR efficacy, thereby relieving the inhibitory tone on dopamine-mediated downstream signaling. To assess the impact of Par-4 loss of function on D2DR efficacy, we sought to silence Par-4 expression using RNA interference (RNAi) against Par-4 in a DNA-based vector (Sui et al., 2002). The Par-4 siRNA effectively knocked down the expression of endogenous Par-4 protein in the HEK93 cells and in cultured rat striatal neurons. We next analyzed the direct effect of Par-4 loss-of-function on D2DR efficacy by measuring D2DR-mediated inhibition of forskolin-activated adenylyl cyclase activity (Sokoloff et al., 1992) in a stable D2DR-EGFP cell line. While D2DR activation by quinpirole, a D2-like dopamine receptor agonist, resulted in a significant decrease in forskolin-activated cAMP accumulation in mock transfected cells, the downregulation of adenylate cyclase was not detectable in Par-4 siRNA transfected cells. Moreover, Par-4 siRNA produced an enhanced dopamine-mediated cAMP accumulation in cultured rat striatal neurons. Taken together, these observations indicate that Par-4 loss-of-function negatively affects D2DR efficacy, thereby relieving the inhibitory tone on dopamine-mediated cAMP signaling.

### Disruption of the Interaction Between PAR-4 and D2DR Results in Upregulation of Dopamine-Mediated Camp Signaling in PAR-4ΔLZ Striatal Neurons

To further test the physiological relevance of the interaction between Par-4 and D2DR in vivo, we employed a deletion mutant mouse, Par-4ΔLZ, that lacks the expression of the C-terminal leucine zipper region of Par-4 responsible for interaction with D2DR (FIGS. 1A and 5A) (Affar et al., 2005). The knockout of exons 4 and 5 in the Par-4 locus by homologous recombination, resulted in expression of the truncated Par-4ΔLZ protein instead of full length Par-4 in the mutant brain extract.

To examine the importance of the Par-4/D2DR interaction in dopamine-cAMP signaling in vivo, we analyzed the dopamine-mediated cAMP accumulation in cultured primary striatal neurons derived from wild type and Par-4ΔLZ embryos. No overt morphological differences were observed between cultured striatal neurons from wild type and Par-4ΔLZ embryos. Remarkably, mutant neurons exhibited a significantly altered response profile of cAMP levels upon treatment with increasing concentrations of dopamine compared to wild type neurons. Specifically, 1-10 μM of dopamine markedly elevated cAMP levels in mutant neurons, indicating a reduced inhibitory tone on dopamine-mediated cAMP signaling. Noteworthy is that this dopamine concentration is within the physiological range of phasic dopamine in the striatum (Jones et al., 1998), as well as the affinity (Kd) of dopamine to mammalian D2DR (Bunzow et al., 1988).

To further delineate the altered cAMP response upon dopamine treatment in Par-4ΔLZ neurons we employed D 1 and D2 antagonists in the assay. When the SCH23390, a D1DR specific antagonist, was co-treated with dopamine, the enhancement of cAMP response in Par-4ΔLZ neurons was abolished, indicating that activation of dopamine D1 receptor (D1DR) underlies the cAMP response at 1-10 μM of dopamine. When dopamine and sulpiride, a D2-specific antagonist, were co-treated in the wild type neurons, the cAMP response was enhanced at the l-10 μM dopamine, which is reminiscent of the increase observed in Par-4ΔLZ neurons. This result indicates that D2DR activity plays a role to form an inhibitory tone on the cAMP system in this concentration range. Notably, D2-specific antagonist revealed no such effect on Par-4ΔLZ neurons, supporting that D2DR function is impaired in these neurons. Based on these results, it is likely that the decreased inhibitory tone caused by impaired D2DR efficacy in Par-4ΔLZ neurons contributes to the concentration-specific upregulation of the cAMP response.

### Dopamine-Dependent CREB Activity is Upregulated in the Striatal Neurons From PAR-4ΔLZ Mice

cAMP-responsive element binding protein (CREB) is a downstream transcription factor whose activity is regulated by the cAMP-PKA signaling pathway. To examine whether the altered dopamine-mediated cAMP signaling in Par-4ΔLZ neurons has further impact on downstream events, we analyzed the phosphorylation status of CREB at serine 133 (S133), a site that is phosphorylated by the cAMP-dependent protein kinase (PIA) in response to dopamine (Gonzalez et al., 1989). In wild type neurons, CREB S133 phosphorylation was significantly decreased upon treatment of dopamine in a dose-dependent manner. Interestingly, the dopamine-induced downregulation of CREB S133 phosphorylation was not observed in Par-4ΔLZ neurons. When compared to the wild type, CREB S133 phosphorylation is markedly upregulated in Par-4ΔLZ neurons, which is consistent with the observed upregulation of dopamine-mediated cAMP accumulation in Par4-ΔLZ neurons. This result suggests that the downstream events of dopamine-mediated cAMP signaling are affected in the absence of Par-4/D2DR interaction.

### PAR-4ΔLZ Mice Show Increased Depression-Like Behaviors

Dysfunction of the mesolimbic dopamine system is one of the leading candidates for the etiology of certain characteristic symptoms of depression such as anhedonia and amotivation (TAP., 1994). As such, we tested whether abnormalities in dopamine-mediated signaling in Par-4ΔLZ mice have physiological consequences related to depression-like behaviors by employing the Porsolt's forced swim test (FST), a well-established behavioral paradigm to detect depression-like behavior in rodents (Porsolt et al., 1977). Enhanced immobility with no attempt to escape in this test reflects a “depressive mood”, as antidepressants were shown to influence this behavior. Remarkably, Par-4ΔLZ mice display elevated immobility scores compared to wild type, hence an increased depression-like behavior. To verify this result, we performed the tail suspension test (TST), in which a rapid adoption of an immobile posture is shown to reflect a “depressive mood” in rodents (Steru et al., 1985). Par-4ΔLZ mice showed significantly elevated immobility scores in the TST compared to wild type mice, confirming increased depression-like behaviors in Par-4ΔLZ mice. We next tested Par-4ΔLZ mice in the novelty-suppressed feeding (NSF) paradigm, which has been effectively used to assess the efficacy of antidepressants by eliciting competing motivations; the drive to eat and the fear of venturing into the open field (Santarelli et al., 2003). In this test, Par-4ΔLZ mice exhibited significantly increased latency to contact food, indicative of a reduced motivation over an aversive environment, a feature of clinical depression. In addition, we analyzed behaviors of Par-4ΔLZ mice in the open field to determine whether the mutant mouse has abnormalities in explorative activity, since reduced activity in the open field has been correlated with depression-like behaviors in rodents (El Yacoubi et al., 2003). Indeed, the total explorative activity of Par-4ΔLZ mice in an open field measured by total distance traveled in the arena was decreased, supporting the depression-like behaviors in Par-4ΔLZ mice.

To examine whether the enhanced depression-like behaviors in Par-4ΔLZ mice is compromised by a potential anxiety-like behavior, we analyzed the ambulatory pattern of the mice in the open field test, in which the center activity has been known to inversely reflect anxiety level (El Yacoubi et al., 2003). The ambulatory pattern and center activities of wild type and Par-4ΔLZ mice were not significantly different, suggesting that anxiety level is not altered in Par-4ΔLZ mice. To further verify this interpretation, we performed the elevated plus maze test, an anxiety-like behavioral test (Lister, 1987). In this test, the fraction of time spent in the open and closed arms of the maze was not significantly different between Par-4ΔLZ and wild type mice, further supporting a normal anxiety level in Par-4ΔLZ mice. In addition, no overt anatomical abnormalities of the adult Par-4ΔLZ mouse brain were detected, and the performance of Par-4ΔLZ mice in a rotarod test is not significantly different from that of wild type mice, indicating that the enhanced depression-like behavior of Par-4ΔLZ mice is not likely due to defects in brain development and/or motor coordination. Collectively, these results show that a disrupted modulation of dopamine signaling caused by loss of Par-4/D2DR interaction in Par-4ΔLZ mice is associated with depression-like behaviors.

### DISCUSSION

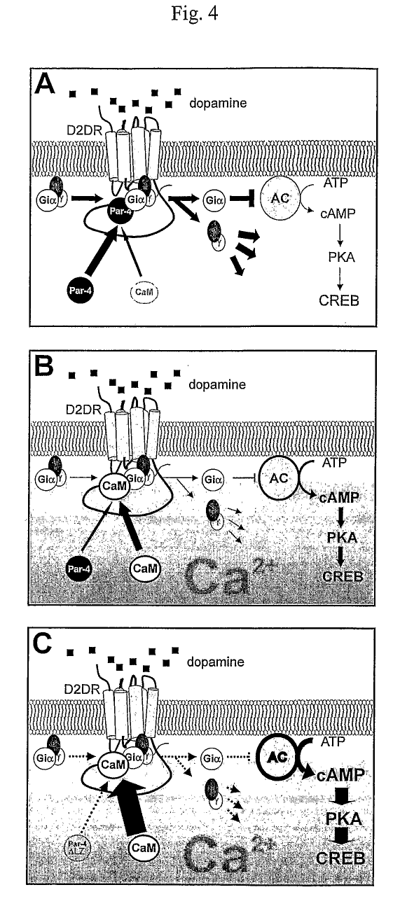

In the present study, we have reported a novel function of Par-4 as a modulatory component in dopamine signaling, demonstrating that Par-4/D2DR complex formation is necessary to maintain a inhibitory tone on dopamine-mediated cAMP signaling generated by D2DR under low Ca²⁺ condition (FIG. 4A). A shift in the equilibrium toward calmodulin/D2DR complex can occur when Ca²⁺-influx activates calmodulin, thereby relieving D2DR-mediated inhibitory tone on cAMP signaling (FIG. 4B). Disruption of Par-4/D2DR interaction in Par-4ΔLZ mice may facilitate calmodulin/D2DR complex formation upon Ca²⁺ influx, hence an upregulation of dopamine-cAMP-CREB signaling, which may contribute to increased depression-like behaviors (FIG. 4C). Thus, identification of Par-4/D2DR interaction potentially reveals a novel mechanism for a cross-talk between Ca²⁺ signaling and dopamine-mediated cAMP signaling.

The physiological relevance of the interaction between Par-4 and D2DR and its modulation of cAMP signaling is signified by depression-like behaviors in Par-4ΔLZ mice. This observation is of particular interest in that there is ample evidence suggesting that impairment of dopamine signaling is involved in the manifestation of depression (Manji et al., 2001; Willner, 1995). For example, anhedonia and amotivation, symptoms prominent in depressive patients, are mainly governed by dopamine neurotransmission in reward and motivation circuits (Nader et al., 1997). Moreover, dopamine metabolites in cerebrospinal fluid are reduced in depressive subjects (Bowden et al., 1997). Conversely, a depressive syndrome is frequently encountered in subjects affected by Parkinson's disease, a nigrostriatal hypodopaminergic disorder (Burn, 2002). Notably, D2DR antagonists can induce ‘pharmacogenic depression’ in schizophrenic patients (Willner, 1995), and chronic treatment with antidepressants produces behavioral sensitization to D2DR agonists (Maj et al., 1996). These observations unequivocally suggest that perturbed D2DR-mediated signaling may underlie the manifestation of depressive symptoms, and that effects of antidepressants also involve an adaptation of D2DR signaling pathways. Nonetheless, underlying molecular mechanisms have not been elucidated. In the present study, we have reported perturbed dopamine signaling in Par-4ΔLZ mice caused by disrupted Par-4/D2DR interaction. Since Par-4ΔLZ mice exhibit depression-like behaviors, it is likely that the perturbed dopamine signaling in Par-4ΔLZ mice may mimic certain aspects of pathological states of depression at molecular levels, such as an altered CREB activity.

Indeed, roles for cAMP-CREB signaling in the pathophysiology of depression and antidepressant action have been suggested by numerous studies. However, the impact of the changes in cAMP-CREB signaling on the manifestation of depression and the outcome of chronic antidepressant treatment is complex. In some studies, the upregulation of cAMP-CREB is casually correlated with chronic antidepressant effects (Chen et al., 2001; Dowlatshahi et al., 1998). On the other hand, there is also evidence that blockade of cAMP-CREB signaling underlies antidepressant-like effects. For example, repeated antidepressant administration decreases levels of CREB phosphorylation in frontal cortex (Manier et al., 2002). Furthermore, inhibition of CREB activity in the nucleus accumbens produces an antidepressant-like effect in animal models of depression whereas overexpression of CREB in this region elicits opposite effects (Newton et al., 2002; Pliakas et al., 2001). Collectively, it appears that the effect of CREB activity on depression-like behaviors is brain region-specific, mediating differential responses to antidepressants in the nucleus accumbens and other brain regions. In the present study, we have demonstrated an upregulation of dopamine-dependent cAMP-CREB signaling in the striatal neurons from Par-4ΔLZ mice in association with depression-like behaviors. This observation is in agreement with reports that an increase in CREB activity in the D2DR-rich nucleus accumbens, a major target of mesolimbic dopaminergic tracts in the striatum, is connected to behavioral responses to emotional stimuli and depressive symptoms (Barrot et al., 2002; Nestler et al., 2002). Thus, the enhanced dopamine-dependent CREB activity and associated changes in gene expression profile in the reward circuits is likely to contribute to depression-like behaviors in Par-4ΔLZ mice.

It is well established that D2DR function is required for normal motor coordination (Viggiano et al., 2003). Interestingly, Par-4ΔLZ mice do not exhibit overt defects in motor skills. We speculate that, by disrupting the direct interaction between D2DR and Par-4, only Par-4-mediated modulatory events in D2DR signaling is impaired in vivo, which may be functionally important only in certain circumstances, such as controlling mood.

The data presented here do not rule out the possibility that the behavioral phenotypes of Par-4ΔLZ mice are due to direct modifications of other systems such as serotonin or norepinephrine neurotransmissions by a similar mechanism. However, such possibility is less likely for the following reasons. First, calmodulin-mediated downregulation of D2DR efficacy is relatively specific (Bofill-Cardona et al., 2000). Second, our interaction study of Par-4 with the long third intracellular loops of related G-protein coupled receptors tested did not reveal any significant interaction, indicating that Par-4 does not interact with GPCRs in a promiscuous manner. Third, a comparable upregulation of cAMP signaling upon treatment of serotonin and norepinephrine in Par-4ΔLZ striatal neurons was not detected. Nevertheless, given the broad expression of Par-4 in the CNS, additional neuronal functions of Par-4 and potential contribution of other neural circuits have yet to be determined.

Thus, we recently discovered a role for the prostate apoptosis response 4 (Par-4) in dopamine signaling. Major findings are the following:

(1) Par-4 is a novel dopamine D2 receptor (D2DR)-interacting protein.

(2) The interaction is mediated by N-terminal 30 aminoacid residues of D2DR 3^{rd} intracellular loop and Par-4 leucine zipper domain.

(2) Par-4/D2DR interaction can be disrupted by Ca²⁺/cahnodulin.

(3) A disruption of Par-4/D2DR interaction causes an upregulation of DA-cAMP-CREB signaling in the striatal neurons due to an impairment of D2DR function.

(4) A genetically engineered mouse (Par-4ΔLZ) with a disrupted Par-4/D2DR association exhibits depression-like behaviors.

Some of the direct implications from the findings in human health are:

(1) Par-4 function mediated by interaction with D2DR is critical for the normal maintenance of mood.

(2) An impairment of Par-4 function may elicit depressive symptoms.

(3) Conversely, an enhancement of Par-4 function in the nervous system may have positive effect on normal mood control.

(4) Small molecules modulating Par-4 function may possess anti-depressant and/or mood stabilizing activity in the patients of mood disorders with high commercial potential.

### EXPERIMENTAL PROCEDURES

In vitro binding assay

pPC97-D2i3 and pPC86-Par-4LZ plasmids were digested with SalI and NotI and cloned into pGEX4T-2 (Amersham-Pharmacia) using the same restriction sites to make GST-D2i3 and GST-Par-4LZ fusion proteins, respectively. GST-fusion proteins were expressed in BL21 bacteria and purified following manufacturer's instruction. For the in vitro binding assay, 500 nmoles of GST-Par-4LZ fusion protein in PBS was digested with 0.2 NIH units of thrombin (Sigma) for 2 hours at room temperature and the reaction was stopped by adding PMSF (10 μM, Sigma) and incubated for an additional 1 hour at 4° C. The GST portion of the digested protein was removed by glutathione sepharose (Amersham-Pharmacia). The supernatant was equilibrated to final 1× binding buffer (200 mM NaCl, 0.2% Triton X-100, 0.2mg/ml BSA and 50 mM Tris, pH 7.5). Binding reaction was initiated by adding 500 nmoles of GST-D2i3 (50 nM of GST-D2i3²²¹⁻²⁴¹ in the competition assay) to Par-4LZ protein in the 1× binding buffer and incubated for 2-3 hours at 4° C. GST-D2i3 was precipitated using 100 μl of 10% glutathione sepharose in 1× binding buffer. The precipitate was washed 3 times with 1× binding buffer and resuspended in 2× SDS sample loading buffer.

### Antibodies

Anti-Par-4 anti-rabbit polyclonal (R334) (Cheema et al., 2003; Duan et al., 1999a), anti-Par-4 anti-mouse monoclonal (A10) (Bieberich et al., 2003), anti-D2DR anti-goat polyclonal (N19) (Scott et al., 2002), anti-rabbit polyclonal (H50) (Dunah et al., 2002), anti-GST rabbit polyclonal and monoclonal antibodies were purchased from Santa Cruz Biotechnology. Anti-rabbit anti-DARPP-32 antibody was from Cell Signaling. Anti-synaptophysin (SVP-38) and anti-α-tubulin monoclonal antibodies were from Sigma. Anti-rabbit anti-GFP antibody and Cy5-conjugated anti-mouse IgG were purchased from Molecular Probes. FITC-conjugated anti-rabbit IgG was purchased from ICN, and Texas red-conjugated anti-goat IgG from Santa Cruz Biotechnology.

### Immunoprecipitation

Mice were euthanized in a C0₂ chamber and the brain was dissected out and dounce-homogenized in the BF2 (150 mM NaCl, 1% NP-40, 0.5% sodium deoxycholate, 0.1% SDS and 50 mM Tris (pH8.0), 5 mM EDTA, 5 mM EGTA, 5 nM glycerol-2-phosphate, 2 mM sodium pyrophosphate, 5 mM NaF, 2 mM Na₃VO₄, 1 mM DTT, phosphatase inhibitor cocktail-I (Sigma), EDTA-free protease inhibitor cocktail (Roche), 10 μM ALLM (Calbiochem)). For coimmunoprecipitation using anti Par-4 antibody, the brain was first homogenized in BF1 (150 mM NaCl, 1% NP-40, 50 mM Tris (pH8.0), EDTA-free protease inhibitor cocktail (Roche), 10 μM ALLM (Calbiochem)), centrifuged for 15 min at 12,000 g, and the pellet was resuspended in BF2 and homogenized. Homogenate was centrifuged (10,000 g) and the supernatant was used for immunoprecipitation. Protein extract was incubated with 1 μg of antibody on a rocking plate for 1 hour at 4° C. 100 μl of 10% protein-G sepharose (Amersham-Pharmacia) in the same lysis buffer was added and incubated for an additional 45 min at 4° C. with gentle shaking. The precipitate was washed three times with lysis buffer and resuspended in 2× SDS sample loading buffer. For D2DR co-immunoprecipitation, anti-D2DR antibodies were conjugated to Dynabeads (DYNAL) following the manufacturer's instruction and incubated with brain lysates. The proteins were eluted in ethanolamine, lyophilized and dissolved in 2× SDS sample loading buffer. For co-immunoprecipitation from the stable D2DR-EGFP cell line, cells cultured to ˜90% confluency in the 10 cm plate were lysed in a lysis buffer (150 mM NaCl, 1% Triton X-100, 5 mM EDTA, 5 mM EGTA, 50 mM Tris (pH8.0), 5 mM glycerol-2-phosphate, 2 mM sodium pyrophosphate, 5 mM NaF, 2 mM Na₃VO₄, 1 mM DTT, phosphatase inhibitor cocktail-I (Sigma), EDTA-free protease inhibitor cocktail (Amersham-Pharmacia), 10 μM ALLM (Calbiochem)) for 30 min with gentle shaking at 4° C. Lysates were dounce-homogenized and centrifuged at 12,000 g for 15 min. Supernatants were used for immunoprecipitation.

### Immunohistochemistry

Mice were anesthetized with avertin and perfused transcardially with PBS, followed by 4% parafolmaldehyde/PBS. Comparable 6 mm thick paraffin coronal brain sections were deparaffinized and rehydrated. Antigen retrieval was performed by microwave irradiation. Sections were incubated with primary antibodies overnight at 4° C. Bound antibodies were detected by standard streptavidin-biotin-peroxidase methods (Vector Laboratories, Burlingame, Calif.). Immunostaining was performed using ant-rabbit anti D2DR antibody, anti-mouse anti-Par-4 (1:100) and anti-rabbit anti-DARPP-32 (1:100) antibodies.

### Immunocytochemistry

DIV 11-14 mouse striatal neurons cultured on coverslips were fixed in cold 4% paraformaldehyde/PBS for 1 hour. Media was replaced with fresh Neurobasal media containing drugs as indicated in figure legends prior to fixation. Coverslips were incubated for 2 hours in the blocking solution (2% goat serum, 1% triton X-100 in PBS) and primary antibodies were incubated for 6-12 hours and secondary antibodies for 2 hours at room temperature in the blocking solution. Anti-rabbit anti-Par-4 polyclonal antibody was used at a dilution of 1:200, anti-D2DR anti-goat polyclonal antibody at 1:200 and anti-synaptophysin anti-mouse monoclonal antibody at 1:300.

cAMP Enzyme-immunoassay (EIA)

DIV 11-14 neurons cultured in 24 well-plates were replaced with neurobasal media supplemented with 10 mM HEPES (pH7.4) containing drugs indicated for 50 min at room temperature, cells were lysed in 200 μl of 0.1M HCl solution for 15 min with gentle shaking and spun in the microcentrifuge tubes. cAMP concentration of the supernatant was measured using the cAMP-Enzyme Immunoassay Kit (Assay Designs) following manufacture's instructions. Concentrations of cAMP were normalized using protein concentrations measured by Biorad Protein Assay System (Bio-rad).

### Forskolin-activated Adenylate Cyclase Activity Assay

D2DR-mediated inhibition of forskolin-stimulated cAMP production was analyzed in a stable HEK293 cell line expressing D2DR-EGFP. Cells cultured in 24-well dishes were preincubated with rolipram (10 μM) for 15min and subsequently treated with forskolin (1 μM) and increasing concentrations of quinpirole as indicated for 20 min at room temperature. cAMP concentration of the cell lysates were measured by cAMP-EIA.

### Behavioral Tests

Porsolt's forced swim test was performed as previously described (Porsolt et al., 1977). Mice were placed in a plexiglass chamber (diameter; 18 cm, height;30 cm) filled with water (8 cm, 25° C.) and immobility (passive floating without hind leg movements) was scored during the 6 min test session. A tail suspension test was carried out as previously described (Steru et al., 1985). A mouse was suspended by the tail to a rod in a shielded chamber. Two blind observers measured the immobility (no foreleg and hindleg movement) during the 6-min test session and the mean values were used for analysis. Novelty-suppressed feeding behavior was carried out as previously described (Santarelli et al., 2003). Mice were deprived of food for 48hr and exposed to the food in a novel context, a white-lit arena (50×35 cm² ) and monitored using TSE Videomot 2 (TSE Systems). The latency to contact food was analyzed. In the open field test, the exploratory behaviors of the mice were monitored in the 50×35cm² white-lit arena for 5 min using the TSE Videomot 2. To analyze center activity, the arena was divided into 16 rectangular areas (4×4) and time spent in the central 4 subdivisions was quantified. Elevated plus maze tests were carried out as previously described (Lister, 1987) using H10-35-EPM system (Coulbourn Instruments). Mice were placed in the center area of the plus maze and their movements were monitored using TSE Videomot 2. Time spent in the open arms, the closed arms and the center area were quantified. Rotarod tests were performed as previously described (Ona et al., 1999) using Economex Rotarod System (Columbus Inc.). Prior to testing, mice were trained in three sessions (15 min each) on the same rotarod over 2 days. Latency to fall was measured at 4-40 rpm with 1%/sec increment in speed.

### Yeast Two Hybrid Screen

A long isoform of D2i3 (amino acid 212-373) was amplified from a human D2DR cDNA clone (IMAGE:2336819, AI692402) by PCR and subcloned into pPC97 vector to make pPC97-D2i3, GAL4 DNA binding domain fusion protein. MaV203 yeast cells were cotransformed with pPC97-D2i3 and human fetal brain cDNA library (GibcoBRL) plasmids cloned in pPC86. Total 3×106 cotransformants were initially screened for growth on Leu-, Trp- and His- media containing 3-amino-1,2,4-triazol (3-AT, 20 mM), subsequently for growth on Ura- media and expression of P-galactosidase activity. The plasmids were isolated from the positives, amplified in DHSa and analyzed by DNA sequencing.

### Primary Culture

Striata were dissected from E15 129/Sv mice or E18 SD rat embryos in the 1× Hank's Balanced Salt Solution (Invitrogen) supplemented with 20 mM HEPES (pH7.2) and treated with trypsin (0.25%, Sigma) and DNase (0.1%, Sigma) for 5-7 min at 37° C. The cells were mechanically dissociated by triturating with a fine polished glass pipette, diluted in Neurobasal media (Invitrogen) supplemented with 10% horse serum and 10 mM HEPES (pH7.2), and plated in a dish coated with poly-D-lysine (Sigma) and laminin (Sigma).

### Stable D2DR-EGFP Cell Line

The D2DR coding sequence was amplified from a D2DR cDNA clone (IMAGE:2336819, AI692402) by PCR and subcloned in pEGFP-N1 (BD Biosciences Clontech) at HindII/EcoR1 sites. HEK293 cells were transfected with the sequence-verified construct and selected for 4 weeks in the media containing 750 μg/ml Geneticin (GibcoBRL). The stable expression of D2DR-EGFP was verified by immunocytochemistry.

### Small Interference RNA Constructs and Transfection

A Par-4 small interference RNA (siRNA) construct was generated using pSilencer siRNA vector following the manufacturer's instruction (Ambion). The sequences of oligonucleotides used are 5′-gatcccgctgcgctcacggctcgtccttcaagagaggacgagccgtgagcgcag ttttttggaaa-3′ (SEQ ID NO: 7) and 5′-gcttttccaaaaaactgcgctcacggctcgtcctctcttgaaggacg agccgtgagcgcagcgg-3′ (SEQ ID NO: 8). The plasmid was amplified in the XL10 bacteria and purified en mass using the Maxi prep plasmid isolation kit (Biorad). HEK293 cells were transfected using lipofectamine 2000 (Invitrogen) and rat striatal neurons were transfected via electroporation using the Nucleofector kit for rat neurons (AMAXA). Knockdown of the Par-4 protein were assessed 48-72 hrs after transfection.

### Enzyme-linked Immuno-sorbent Assay (ELISA)

Cultured DIV12-13 mouse neurons were lysed for 30 min at 4° C. with gentle shaking in the extraction buffer (50 mM Tris pH8.0, 50 mM NaCl, 1.0% NP-40, 0.5% sodium deoxycholate, 0.1% SDS, 1 mM EDTA, 1 mM EGTA, 1 mM NaF, 20 mM Na4P2O7, 2 mM Na3VO4, 10% glycerol) supplemented with protease inhibitor cocktail (Sigma). Cell lysates were centrifuged for 15 min at 12,000 g at 4° C. and the supernatants were applied to ELISA using the Immunoassay kits for total CREB and S133 phospho-CREB (Biosource) following the manufacturer's instruction.

### REFERENCES

Affar et al. (2005) in preparation. Baldessarini, R. J. (1989) J Clin Psychiatry 50, 117-126.
Barrot et al. (2002) Proc Natl Acad Sci U S A 99, 11435-11440. Bieberich et al. (2003) J Cell Biol 162, 469-479. Blazer et al. (1994) Am J Psychiatry 151, 979-986. Bofill-Cardona et al. (2000) J Biol Chem 275, 32672-32680. Bowden, C. et al. (1997) Brain Res 769, 135-140. Bunney et al. (1965) Arch Gen Psychiatry 13, 483-494. Btnzow et al. (1988) Nature 336, 783-787. Bum, D. J. (2002) European Journal of Neurology 9, 44-54. Cheema et al. (2003) J Biol Chem 278, 19995-20005. Chen et al. (2001) Biol Psychiatry 49, 753-762.
Coppen, A. (1967 Br J Psychiatry 113, 1237-1264. De Camilli et al. (1979) Nature 278, 252-254. Delay et al. (1952) Ann Med Psychol (Paris) 110, 689-692. Diaz-Meco et al. (1999) J Biol Chem 274, 19606-19612. Diaz-Meco et al. (1996) Cell 86, 777-786.
Dowlatshahi et al. (1998). Lancet 352, 1754-1755. Duan et al. (1999a) J Neurochem 72, 2312-2322. Duan et al. (1999b) AnnNeurol 46, 587-597. Dunah et al. (2002) Science 296, 2238-2243. El Yacoubi et al. (2003) Proc Natl Acad Sci U S A 100, 6227-6232. Fishburn et al. (1995) J Biol Chem 270, 29819-29824. Fuller, R. W. (1995) Prog Drug Res 45, 167-204. Gonzalez et al. (1989) Nature 337, 749-752. Greenberg et al. (1993) J Clin Psychiatry 54, 405-418. Guo et al. (1998) Nat Med 4, 957-962. Hersch et al. (1995) J Neurosci 15, 5222-5237. Jarvie et al. (1989) J Biochem (Tokyo) 106, 17-22. Johnstone et al (1996) Mol Cell Biol 16, 6945-6956. Jones et al. (1998) J Neurosci 18, 1979-1986. Kinney, J. L. (1985) Clin Pharm 4, 625-636. Kuhn, R. (1958) Am J Psychiatry 115, 459-464. Leonard, B. E. (1978) Acta Psychiatr Belg 78, 770-780. Lister, R. G. (1987) Psychopharmacology (Berl) 92, 180-185. Lytton, J. et al. (1991) J Biol Chem 266, 17067-17071. Maj et al. (1996) Eur J Pharmacol 304, 49-54. Manier et al. (2002) J Neural Transm 109, 91-99. Manji et al. (2001) Nat Med 7, 541-547. Missale et al. (1998) Physiol Rev 78, 189-225. Murer et al. (2002) Cell Mol Neurobiol 22, 611-632. Nader et al. (1997) Annu Rev Psychol 48, 85-114. Nestler, E. J. (2001) Am J Addict 10, 201-217. Nestler et al. (2002) Neuron 34, 13-25. Newton et al. (2002) J Neurosci 22, 10883-10890. Ona et al. (1999) Nature 399, 263-267. Ouimet et al. (1998) Brain Res 808, 8-12. Pedersen et al. (2000) Faseb J 14, 913-924. Pliakas et al. (2001) J Neurosci 21, 7397-7403. Porsolt et al. (1977) Nature 266, 730-732. Santarelli et al. (2003) Science 301, 805-809. Schildkraut et al. (1965) J Psychiatr Res 3, 213-228. Scott et al. (2002) Proc Natl Acad Sci U S A 99, 1661-1664. Sells et al. (1994) Cell Growth Differ 5, 457-466. Sokoloff, P. et al. (1992) Eur J Pharmacol 225, 331-337. Steru et al. (1985) Psychopharmacology (Berl) 85, 367-370. Sui et al. (2002) Proc Natl Acad Sci U S A 99, 5515-5520. TAP., A. (1994) In Diagnostic and Statistical Mannual of Mental Disorders, 4th Ed. (Washington, DC, American Psychiatric Press). Viggiano et al. (2003) Neurosci Biobehav Rev 27, 623-637. Voss et al. (1993) J Biol Chem 268, 4637-4642. Willner, P. (1995) In Psychopharmacology; The Fourth Generation of Progress, F. E. Bloom, and D. I. Kupfer, eds. (Raven, New York), pp. 921-932. Wong, M. L., and Licinio, J. (2001) Nat Rev Neurosci 2, 343-351.

### Example 2

### Valproate Stimulates Par-4 Expression

We have shown that Par-4 is involved in dopamine D2 receptor-medicated signaling and plays an important role in normal mood maintenance. Disruption of Par-4 function is associated with depression-like behaviors in mice. We further investigated if Par-4 function can be modulated by currently available medications for mood disorders. Valproate is one of the most prescribed drugs for bipolar disorder patients. However the exact target of its mood stabilizing effect is currently unknown.

We found that the Par-4 protein level was upregulated in cultured mouse and rat neurons when treated with 1 mM valproate. The induction was most prominent in hippocampal neurons but not restricted to hippocampus as we observed less prominent induction in striatal neurons and cortical neurons. The induction is at least partially mediated by transcription of the Par-4 gene, as we observed an increase in Par-4 mRNA level measured by semi-quantitative reverse transcriptionpolymerase chain reaction (RT-PCR). Moreover, the dopamine signaling measured by cAMP enzyme immunoassay is also altered by valproate treatment in the cultured striatal neurons, which can be easily explained by upregulated dopamine D2 receptor function as a results of increased Par-4 functionality by valproate.

Based on these results it is possible to speculate that valproate transcriptionally induces Par-4 gene to affect dopamine D2 receptor function, which could be a physiological consequence of valproate treatment connected to mood stabilizing process in the bipolar patients.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method for identifying an agent that modulates the interaction between Par-4 and the dopamine D2 receptor (D2DR), comprising:
(i) contacting a Par-4 protein, or a portion thereof that is sufficient for interacting with a D2DR protein, with a D2DR protein, or a portion thereof that is sufficient for interacting with a Par-4 protein, in the presence of a test agent; and
(ii) determining the level of interaction between the Par-4 protein or portion thereof and the D2DR protein or portion thereof, wherein a different level of interaction between the Par-4 protein or portion thereof and the D2DR protein or portion thereof in the presence of the test agent relative to the absence of the test agent indicates that the test agent is an agent that modulates the interaction between Par-4 and D2DR; or
(i) contacting a cell comprising a Par-4 protein or a portion thereof that is sufficient for interacting with a D2DR protein and a D2DR protein or a portion thereof that is sufficient for interacting with a Par-4 protein with a test agent; and
(ii) determining the level of cAMP accumulation or dopamine-dependent cAMP-CREB signaling wherein a different level of cAMP accumulation or dopamine-dependent cAMP-CREB signaling in the presence of the test agent relative to the absence of the test agent indicates that the test agent is an agent that modulates the interaction between Par-4 and D2DR; or a method for identifying an agent that changes the cellular location of Par-4 in a cell comprising
(i) contacting a cell expressing a Par-4 protein or a portion thereof in a first cellular compartment with a test agent; and
(ii) determining the cellular location of the Par-4 or a portion thereof at a certain time after the beginning of the contacting step; wherein a different cellular location of the Par-4 or a portion thereof protein in a cell that was contacted with the test agent relative to a cell that was not contacted with the test agent or relative to the cell before contacting it with the test agent indicates that the test agent is an agent that changes the cellular location of Par-4 in a cell.

2-5. (canceled)

6. The method of claim 1, wherein the cell comprises a heterologous nucleic acid encoding the Par-4 protein or portion thereof and/or a heterologous nucleic acid encoding the D2DR protein or portion thereof.

7-13. (canceled)

14. The method of claim 1, further comprising determining the effect of the test agent on the inhibitory tone of D2DR on dopamine-mediated downstream signaling.

15-22. (canceled)

23. A composition or an isolated molecular complex comprising an isolated Par-4 protein, or a portion thereof that is sufficient for interacting with a D2DR protein, and an isolated D2DR protein, or a portion thereof that is sufficient for interacting with a Par-4 protein.

24. The composition of claim 23, further comprising a test agent.

25. (canceled)

26. An animal model for a Par-4 related disease, consisting of an animal having a mutation in the gene encoding the Par-4 protein, which mutation prevents the encoded Par-4 protein from interacting with the D2DR protein.

27. The animal model of claim 26, wherein the Par-4 protein has a deletion in its leucine zipper region rendering it inactive.

28. (canceled)

29. The animal model of claim 26, wherein the animal is a mouse.

30. A method for increasing the inhibitory tone on dopamine-mediated downstream signaling in a cell comprising a D2DR protein, comprising increasing the level or activity of Par-4 in the cell.

31. The method of claim 30, wherein the cell is a neuron.

32. The method of claim 30, further comprising reducing the level of calcium in the cell.

33. The method of claim 30 for treating a hypo-active Par-4 related disorder in a subject comprising administering to a subject in need thereof an agent that increases the level or activity of Par-4 in cells comprising a D2DR; increases the interaction between Par-4 and D2DR and/or prevents the nuclear translocation of Par-4 in cells.

34. The method of claim 33, wherein the disorder is depression, a depression-like behavior, Parkinson's disease, biopoloar disease, disthymia, eating disorders, restless leg syndrome or hypertension.

35. The method of claim 34, further comprising administering to the subject an agent that reduces the level of calcium in the cell or prevents the level of calcium in the cell to increase to levels contributing to relieving the inhibitory tone on dopamine-mediated downstream signaling.

36. The method of claim 30, comprising introducing into the cell a Par-4 protein or portion thereof or a nucleic acid encoding such.

37-38. (canceled)

39. A method for treating a hyper-active Par-4 related disorder in a subject comprising administering to a subject in need thereof Use of an agent that decreases the level or activity of Par-4 in cells comprising a D2DR; decreases the interaction between Par-4

40. The method of claim 39, wherein the disorder is schizophrenia, schizoaffective disorder, attention deficit hyperactivity disorder (ADHD), Tourette syndrome or drug addition.

41. The method of claim 40, further comprising administering to the subject an agent that increases the level of calcium in the cell or prevents the level of calcium in the cell to decrease to levels contributing to increasing the inhibitory tone on dopamine-mediated downstream signaling.

42. A method for determining whether a subject has or is likely to develop a hypo-active Par-4 disorder, comprising determining the cellular location of Par-4 in a neuron of the subject, wherein the presence of Par-4 in the nucleus of the neuron indicates that the subject has or is likely to develop a hypo-active Par-4 disorder.

43. The method of claim 33, wherein the agent is a compound of formula I, wherein formula I is represented by: 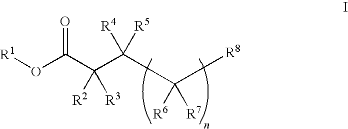
or a pharmaceutically acceptable salt thereof,
wherein,
R¹ is H, alkyl, heteroalkyl, allyl, aryl, or aralkyl;
R², R⁴, and R⁶each represent independently for each occurrence H, alkyl, heteroalkyl, allyl, aryl, aralkyl, halogen, hydroxyl, alkoxy, —N(R⁹)₂, —C(O)R⁹, —OC(O)R⁹, —CO₂R⁹, —C(O)N(R⁹)₂, or —N(R⁹)C(O)R⁹;
R³, R⁵, R⁷, and R⁸ each represent independently for each occurrence H, alkyl, heteroalkyl, allyl, aryl, aralkyl, or alkoxy;
R⁹ represents independently for each occurrence H, alkyl, aryl, or aralkyl;
n is 1, 2, 3, 4, 5, 6, 7, or 8; and
provided that at least one of R², R³, R⁴ or R⁵ is alkyl.


SEQUENCE LISTING (6 sequences)

SEQ ID NO: 1 - DNA, 1967 bp, homo sapiens
  CDS (287)..(1309)
ctgtcctgggattgcctggagctccgcaccgcgagtttgccgcggcactttccgcgcggcggaagagcgcgcgccagcttcggcacacctgggagccggatcccagccctacgcctcgtcccctacaagctcctccaagccccgccggctgctgtgggagcggcggccgtccctctcctggaggtcgtctcctggcatcctcggggccgcaggaaggaagaggaggcagcggccggagccctggtgggcggcctgaggtgagagcccgaccggcccctttgggaatatggcgaccggtggctaccggaccagcagcggcctcggcggcagcaccacagacttcctggaggagtggaaggcgaaacgcgagaagatgcgcgccaagcagaaccccccgggcccggcccccccgggagggggcagcagcgacgccgctgggaagccccccgcgggggctctgggcaccccggcggccgccgctgccaacgagctcaacaacaacctcccgggcggcgcgccggccgcacctgccgtccccggtcccgggggcgtgaactgcgcggtcggctccgccatgctgacgcgggcggcccccggcccgcggcggtcggaggacgagcccccagccgcctctgcctcggctgcaccgccgccccagcgtgacgaggaggagccggacggcgtcccagagaagggcaagagctcgggccccagtgccaggaaaggcaaggggcagatcgagaagaggaagctgcgggagaagcggcgctccaccggcgtggtcaacatccctgccgcagagtgcttagatgagtacgaagatgatgaagcagggcagaaagagcggaaacgagaagatgcaattacacaacagaacactattcagaatgaagctgtaaacttactagatccaggcagttcctatctgctacaggagccacctagaacagtttcaggcagatataaaagcacaaccagtgtctctgaagaagatgtctcaagtagatattctcgaacagatagaagtgggttccctagatataacagggatgcaaatgtttcaggtactctggtttcaagtagcacactggaaaagaaaattgaagatcttgaaaaggaagtagtaagagaaagacaagaaaacctaagacttgtgagactgatgcaagataaagaggaaatgattggaaaactcaaagaagaaattgatttattaaatagagacctagatgacatagaagatgaaaatgaacagctaaagcaggaaaataaaactcttttgaaagttgtgggtcagctgaccaggtagaggattcaagactcaatgtggaaaaaatattttaaactactgattgaatgttaatggtcaatgctagcacaatattcctatgctgcaatacattaaaataactaagcaagtatatttatttctagcaaacagatgtttgttttcaaaatacttctttttcattattggttttaaaaaagcattatccttttatctcacaaataagtaatatctttcagttattaaatgatagataatgcctttttggttttgtgtggtattcaactaatacatggtttaaagtcacagccgtttgaatatattttatcttggtagtacattttctcccttaggaatatacatagtctttgtttacatgagttcaaatacttttgggatgttaccttcacatgtcctattactgatgtgtgcaaccttttatgtgttgatgactcactcataaaggtttttgtctactgtcatttgttctttccacttattctaagcatttagagtaatagagtcatacttttttataacagcaaccttttaaaaggaaagctcttataaagtcactgtcatgttttagttgactaaatataaatttaagagaatacttgaattgtgctatagtaaataaaaatttactattttgtgtttgaaaaaaaaaaaaaaaaaa

SEQ ID NO: 2 - protein, 340 aa, homo sapiens
MATGGYRTSSGLGGSTTDFLEEWKAKREKMRAKQNPPGPAPPGGGSSDAAGKPPAGALGTPAAAAANELNNNLPGGAPAAPAVPGPGGVNCAVGSAMLTRAAPGPRRSEDEPPAASASAAPPPQRDEEEPDGVPEKGKSSGPSARKGKGQIEKRKLREKRRSTGVVNIPAAECLDEYEDDEAGQKERKREDAITQQNTIQNEAVNLLDPGSSYLLQEPPRTVSGRYKSTTSVSEEDVSSRYSRTDRSGFPRYNRDANVSGTLVSSSTLEKKIEDLEKEVVRERQENLRLVRLMQDKEEMIGKLKEEIDLLNRDLDDIEDENEQLKQENKTLLKVVGQLTR

SEQ ID NO: 3 - DNA, 2643 bp, homo sapiens
  CDS (166)..(1497)
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 4 - protein, 443 aa, homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 5 - DNA, 2556 bp, homo sapiens
  CDS (166)..(1410)
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 6 - protein, 414 aa, homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC